(19) United States
(12) Patent Application Publication
(10) Pub. No.: US 2007/0248563 A1
(43) Pub. Date: Oct. 25, 2007

(54) Skin care compositions including marine extracts

(76) Inventors: Carl F. Iovanni, Boston, MA (US); Michael S. Alexiou, Brookline, MA (US)

Correspondence Address: FISH & RICHARDSON P.C., P.O. BOX 1022, MINNEAPOLIS, MN, 55440-1022, US

(21) Appl. No.: 11/406,930

(22) Filed: Apr. 19, 2006

(51) Int. Cl. A61K 36/02; A61K 8/97

(52) U.S. Cl. 424/74; 424/195.17

(57) ABSTRACT

Skin care products, such as depilatories and epilatories, including marine extract blends are provided. The skin care products include one, two, or more of the following marine extracts, in any combination: Enteromorpha Compressa extract, Fucus Vesiculosus extract, Porphyra Umbilicalis extract, Undaria Pinnatifida extract, and Lithothamnium Calcareum extract.

### TECHNICAL FIELD

This invention relates to skin care compositions, and more particularly to depilatory and/or epilatory compositions.

### BACKGROUND

Non-razor based methods for removal of hair from skin, e.g., for cosmetic purposes, include the use of depilatory and/or epilatory products. A depilatory removes hair on the skin's surface, while an epilatory removes the hair from below the skin's surface, optionally with removal of the hair follicle.

Common epilatory products include waxes, which typically work by encasing and grasping hair, allowing the hair to be pulled from the skin when the wax is removed. There are generally two types of waxing techniques. The first is hot waxing, in which a wax is heated to a temperature at which it has a flowable consistency and is applied to the area of skin to be treated. The wax flows around hair on the area of skin being treated and cools, encasing the hair within the wax. The wax, once cool, can be pulled off of the skin, removing the encased hair with the wax and often removing the hair follicle as well. The second method is cold waxing, in which a wax that is at least somewhat flowable at room temperature is applied to an area of skin to be treated, where the wax sticks to (e.g., encases) hair to be removed. The cold wax can be pressed against the skin to assist in encasement of the hair. The cold wax is then removed, taking encased hair and optionally hair follicles along with it. Generally, any area of skin can be waxed, including the upper lip, chin, eyebrows, legs, and bikini line.

Depilatories typically work by reducing the tensile strength of hair (e.g., by breaking chemical bonds within a hair shaft) such that the hair can be easily broken off and washed away. Depilatory products generally contain one or more depilatory chemicals that react with the protein structure of hair, breaking it down. Depending on the coarseness of hair, this process works within 5 to 15 minutes, and the hair is dissolved into a semi-viscous mass, which can then be washed away.

### SUMMARY

Generally, this invention is based on the incorporation of one or more marine extracts into skin care products, e.g., depilation and/or epilation products, to impart beneficial qualities to the skin care products. For example, epilatory and/or depilatory products are provided that include one, two, or more marine extracts, as are skin care products designed for use just prior to or just after the use of an epilatory and/or depilatory product. The inclusion of one or more marine extracts into such products can result in a lessening of and/or relief from the irritation and/or damage that can result from the use of epilatory and/or depilatory products.

In general, the invention features compositions including a depilatory or epilatory agent and one or more marine extracts selected in any combination from the group of Enteromorpha Compressa extracts, Fucus Vesiculosus extracts, Porphyra Umbilicalis extracts, Undaria Pinnatifida extracts, and Lithothamnium Calcareum extracts. These compositions can include any combination of two, three, four, or all five of these extracts, and the extracts, in combination, can be present at a level of at least about 10 ppm or 25 ppm.

In these compositions, the depilatory agent can include a thioglycolic acid salt or a potassium thioglycolate. The pH of the compositions can vary from about 10.0 to about 12.6. The depilatory agent can be or include an alkali stannite. The compositions can further include urea, one or more pigments, fragrances, viscosity modifiers, and/or moisturizers that are not a marine extract. The compositions can also include one or more cross-polymers such as poly(methylvinylether/maleic anhydride) decadiene. The epilatory agent can be a wax, such as a sugar-based wax, e.g., including dextrose. In certain embodiments, the wax can include a hydrogenated rosinate.

In various embodiments, each marine extract is present in the compositions at a level of at least about 0.5 ppm, and the marine extracts, in combination, can be present at a level of at least about 1 ppm or about 2.5 ppm.

In another aspect, the invention features epilatory compositions that include a marine extract blend including one or more of an Enteromorpha Compressa extract, a Fucus Vesiculosus extract, a Porphyra Umbilicalis extract, a Undaria Pinnatifida extract, and a Lithothamnium Calcareum extract; a sugar wax; and glycerin. These epilatory compositions can include from about 0.005% to about 0.02% by weight marine extract blend, from about 75% to about 90% by weight dextrose, and from about 5% to about 15% by weight glycerin. The marine extract blends in the compositions can include between about 0.5% and about 1% by weight of any one or any combination of two, three, four, or all five of an Enteromorpha Compressa extract, a Fucus Vesiculosus extract, a Porphyra Umbilicalis extract, an Undaria Pinnatifida extract, and a Lithothamnium Calcareum extract and a diluent.

In certain embodiments, the marine extract blends in these compositions further include a diluent, such as one or more mineral oils and/or one or more antioxidants.

In another aspect, the invention includes depilatory compositions that include a marine extract blend including one or more (in any combination) of an Enteromorpha Compressa extract, a Fucus Vesiculosus extract, a Porphyra Umbilicalis extract, a Undaria Pinnatifida extract, and a Lithothamnium Calcareum extract; a thioglycolate salt; urea; sodium silicate; and water. In certain embodiments, the compositions include from about 0.05% to about 0.2% by weight marine extract blend, from about 10% to about 20% by weight thioglycolate salt, from about 2% to about 8% urea, and from about 2% to about 8% sodium silicate. These compositions can further include from about 1% to about 5% of a cross-polymer. In certain embodiments, the marine extract blend within the composition includes between about 0.5% and about 1% by weight of each of the five extracts and a diluent. The compositions further include glycerin.

In another aspect, the invention features compositions including a diluent and a marine extract blend including at least two of the five extracts described herein. The marine extract blends can include any two, three, or four extracts in any combination, or all five of the extracts. The marine extract blends in the compositions can include between about 0.5% and about 1% by weight of each of the five extracts, and the diluent can be or can include mineral oil, water, glycerin, or any combination. The compositions can further include an antioxidant and/or a preservative.

In these compositions, the diluent can be a cleaning agent, such as alcohol, and can further include a numbing agent, such as an aromatic anesthetic or a salicylate. The aromatic anesthetic can be, e.g., menthol, eucalyptol, menthyl lactate, menthone glycerol ketal, WS-23 (2-Isopropyl-N,2,3-trimethylbutyramide), WS-3 (N-Ethyl-p-menthane-3-carboxamide), or WS-5 [Ethyl 3-(p-menthane-3-carboxamido)acetate]. The salicylate can be, e.g., methyl salicylate or sodium salicylate.

In these compositions, the Enteromorpha Compressa extract can include an oligosaccharide, the Fucus Vesiculosus extract can include an alginate and organic iodine, the Porphyra Umbilicalis extract can include an amino acid, the Undaria Pinnatifida extract can include a peptide and a polysaccharide, and the Lithothamnium Calcareum extract can include a mineral salt.

In other aspects, the invention also includes methods of making the compositions described herein, by obtaining the marine extracts; obtaining a diluent; and mixing the marine extracts into the diluent at a temperature below 90° C. The invention also includes methods of soothing the skin by applying the compositions described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### DETAILED DESCRIPTION

This invention stems from the discovery that the inclusion of certain marine extracts in skin care products, e.g., depilatory and/or epilatory products, can result in a more pleasant experience for the consumer, with beneficial effects to the skin provided by the marine extracts. For example, in many instances, the use of an epilatory and/or depilatory product can cause irritation and/or damage to the skin being treated. The inclusion of marine extracts in the epilatory and/or depilatory product, or in a skin care product used just prior to or just after the use of an epilatory and/or depilatory product, can result in a lessening of and/or relief from the irritation and/or damage.

Marine Extracts

Generally, one or more marine extracts are incorporated into a skin care product, e.g., an epilatory and/or depilatory product. Exemplary marine extracts include:

| Extract | Active | Function |
|---|---|---|
| Entermorpha Compressa | Oligosaccharides | Restores ion balance, |
| | | refreshing |
| Fucus Vesiculosus | Organic iodine, | Soothing |
| | Alginates | |
| Porphyra Umbilicalis | Amino acids | Moisturizing |
| Undaria Pinnatifida | Peptides, | Nutrition, moisturization |
| | Polysaccharides | |
| Lithothamnium Calcareum | Mineral salts | Provides minerals, free- |
| | | radical scavengers |

Entermorpha Compressa Oligosaccharides Restores ion balance, refreshing Fucus Vesiculosus Organic iodine, Alginates Soothing Porphyra Umbilicalis Amino acids Moisturizing Undaria Pinnatifida Peptides, Polysaccharides Nutrition, moisturization Lithothamnium Calcareum Mineral salts Provides minerals, free-radical scavengers Entermorpha Compressa belongs to a group of algae that represents the main component of the flora from marine areas of large changes in one or more of salinity, temperature, or nutrient concentration. Entermorpha Compressa is made up largely of carbohydrates (generally between about 29 wt % and 40 wt %), proteins (generally between about 9 wt % and 21 wt %) and lipids (generally between about 3.4 wt % and about 4.4 wt %), with the weight percentages based on the dry weight of the algae. A portion of the carbohydrates is in the form of oligosaccharides. Various vitamins (e.g., vitamin C) and minerals (e.g., sodium, potassium, magnesium, manganese, iron, iodine, and calcium) are found in low levels. The extract of Entermorpha Compressa is believed to aid in the ion-regulation of the skin, e.g., regulating K⁺, Na⁺, and/or Ca²⁺ levels in the skin. The extract also helps moisturize the skin and act as a refreshing agent, in that it provides a cooling sensation to the skin.

Fucus Vesiculosus is an edible brown kelp known familiarly as bladderwrack, and is common in the North Atlantic south to the Canary Islands. Fucus Vesiculosus contains a wide spectrum of polysaccharides including fucoidans and fucans. In general, fucoidans are a family of high molecular weight sulfated polysaccharides, widely dispersed in the cell walls of brown seaweed. Fucoidans are believed to contribute a soothing property, as it is believed that fucoidans possess anti-inflammatory activity and possibly anticoagulant effects. Fucoidans may also impart antibacterial and/or antiviral properties. Porphyra Umbilicalis, known also as Nori, purple layer, or sloke, is a small red alga with an irregularly shaped, broad frond that is membranous, but tough. Porphyra Umbilicalis has a high protein content (25-35% of dry weight), and also possesses vitamins and mineral salts, especially iodine. Its vitamin C content is about 1.5 times that of oranges. It moisturizes the skin when topically applied.

Undaria Pinnatifida, also known as Wakame or Japanese kelp, is a brown macroalgae that contains various sulphated polyanions. Undaria Pinnatifida provides nutrition to the skin, and moisturizes the skin when topically applied.

Lithothamnium Calcareum is a calcareous alga having high amounts of minerals. Its skeleton is mainly composed of carbonated calcium and carbonated magnesium, with the two elements representing about 35% of the plant (dry weight). Lithothamnium Calcareum also contains significant levels of other minerals and trace elements like phosphorous, potassium, manganese, boron, iodine, zinc, copper, selenium, and cobalt. Lithothamnium Calcareum thus provides minerals and free-radical scavengers to the skin.

In some embodiments, marine extracts from seaweeds related to any of Enteromorpha Compressa, Fucus Vesiculosus, Porphyra Umbilicalis, Undaria Pinnatifida, or Lithothamnium Calcareum can be employed. For example, extracts of other members of the Enteromorpha genus (e.g., Enteromorpha intestinalis, Enteromorpha linza, and Enteromorpha prolifera), the Fucus genus (e.g., Fucus spiralis and Fucus serratus), the Porphyra genus (e.g., Porphyra yezoensis, Porphyra vietnamensis, Porphyra naiadum, and Porphyra tenera), the Undaria genus (e.g., Undaria undarioides and Undariella peterseniana), and/or the Lithothamnium genus (e.g., Lithothamnion corallioides) can be employed, either alone or in combination, in skin care products.

Suitable individual marine extracts are commercially available, e.g., from Provital, S.A. (Barcelona, Spain). The extracts are typically produced using standard extracting techniques, and contain approximately 95 percent by weight or more of a carrier, such as water, glycerin, caprylic/capric triglyceride, or mineral oil. The extract also contains about 2.5 to about 5.0 percent by weight of the marine extract or extract blend. The extract can also optionally include less than about 0.5 percent by weight, e.g., 0.1 or 0.2 percent by weight of an antioxidant, such as potassium sorbate and/or sodium benzoate (when the carrier is aqueous) or glycine soja (soybean) oil or rosemary leaf extract (when the carrier is non-aqueous).

Marine Extract Blends

In some embodiments, a blend of marine extracts is created. This marine extract blend can be subsequently added to a skin care product, e.g., a depilatory and/or epilatory product, to impart beneficial properties to the skin being treated. The marine extract blend can include two or more (e.g., three, four, or five or more) different marine extracts, for example, from those listed above, to impart multiple beneficial properties to the skin care product. In some embodiments, the marine extracts are present in roughly equal amounts. For example, in some embodiments, each extract is present at a level of no less than about 50% (e.g., no less than about 60%, 70%, 80%, 90%, or 95%) of the level of any other marine extract.

Generally, the marine extract blend will include a diluent or carrier into which the marine extracts are dissolved and/or dispersed. The diluent can in some embodiments be water-based or oil-based, for example, water, glycerin, water-glycerin mixtures, and/or mineral oil. In some embodiments, each marine extract is present in the marine extract blend at levels of at least about 0.1% (e.g., at least about 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, or 1.9%) and/or at most about 2.0% (e.g., at most about 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, or 0.2%) by weight. The total level of marine extracts amounts to about 2.0 to 5.0 percent by weight of the total composition.

Additional ingredients can be incorporated into the marine extract blend, for example, to aid in processing or admixing the marine extract blend, to permit the marine extract blend to resist degradation (e.g., to improve the shelf-life of the extracts in the marine extract blend and/or in the final product), or to provide additional skin-benefiting agents to the marine extract blend. Exemplary ingredients include non-marine-extract moisturizers, refreshing agents, minerals, nutrients, and/or free-radical scavengers, antioxidants (e.g., soybean oil, rosemary leaf extract, Vitamin E and its derivatives (e.g., Tocopheryl Acetate), Vitamin C and its derivatives (e.g., ascorbyl palmitate, ascorbyl stearate, calcium ascorbate, sodium ascorbyl phosphate), BHA (butylated hydroxyanisole), BHT (butylated hydroxytoluene), and/or pentaerithrityl tetra-di-t-butyl hydroxyhydrocinnamate,), preservatives (e.g., potassium sorbate, sodium benzoate, benzylparaben, methylparaben, propylparaben, isobutylparaben, isopropylparaben, phenylparaben, and/or DMDM hydantoin), fragrances, colorants, and processing aids.

Skin Care Products

The marine extracts and marine extract blends are in certain embodiments incorporated into a skin care product, such as, for example, a depilatory and/or epilatory composition and/or a wipe, cream, lotion, or gel that is used pre- or post-depilation and/or epilation. In certain embodiments, the skin care product includes two or more marine extracts (e.g., three or more, four or more, or five or more marine extracts). In certain embodiments including multiple marine extracts, each individual marine extract is present at levels of at least about 1 ppm (e.g., at least about 5 ppm, 10 ppm, 50 ppm, 100 ppm, 500 ppm, 1000 ppm, 2000 ppm, 5000 ppm, 10,000 ppm, or 50,000 ppm) by weight and/or at most about 100,000 ppm (e.g., at most about 50,000 ppm, 10,000 ppm, 5000 ppm, 2000 ppm, 1000 ppm, 500 ppm, 100 ppm, 50 ppm, 10 ppm, or 5 ppm) by weight of the skin care product.

In some embodiments, the marine extracts are present in roughly equal amounts. For example, in some embodiments, each extract is present at a level of no less than about 50% (e.g., no less than about 60%, 70%, 80%, 90%, or 95%) of the level of any other marine extract.

The skin care product can in certain embodiments have a total level of marine extract (inclusive of all marine extracts in the skin care product) of at least about 2 ppm (e.g., at least about 5 ppm, 10 ppm, 50 ppm, 100 ppm, 500 ppm, 1000 ppm, or 5000 ppm) by weight and/or at most about 10,000 ppm (e.g., at most about 5000 ppm, 1000 ppm, 500 ppm, 100 ppm, 50 ppm, 10 ppm, or 5 ppm) by weight.

In certain embodiments, the skin care product includes a marine extract blend. The marine extract blend can be present in the skin care product at levels of at least about 0.02% (e.g., at least about 0.04%, 0.06%, 0.08%, 0.10%, 0.12%, 0.14%, 0.16%, 0.18%, 0.20%, 0.25%, 0.3%, 0.4%, 0.5%, 0.6%, 0.7%, 0.8%, 0.9%, or 1.0%) by weight of the final product and/or at most about 2.0% (e.g., at most about 1.0%, 0.9%, 0.8%, 0.7%, 0.6%, 0.5%, 0.4%, 0.3%, 0.25%, 0.2%, 0.18%, 0.16%, 0.14%, 0.12%, 0.10%, 0.08%, 0.06%, or 0.04%) by weight of the skin care product.

Waxes

In certain embodiments, the skin care product to which one or more marine extracts or marine extract blends are added is a depilatory and/or epilatory wax (e.g., a sugar-based wax, a honey wax, or a cream wax). In certain embodiments, the wax can be a hot wax (sometimes referred to as a pot wax), which is typically hard to medium hard and non-flowable at room temperature, and more fluid-like (e.g., free flowing) when heated to a sufficient temperature (e.g., from about 100° F. to about 150° F.). Hot waxes are typically designed for professional use. In other embodiments, the wax can be a warm wax, e.g., a warm sugar wax, while in still other embodiments, the wax can be a cold wax, which is typically gummy at room temperature, and is generally used without heating or with minimal heating. Warm and cold waxes are generally suitable for home use.

In some embodiments, the wax can include hydrogenated rosinates (e.g., triethylene glycol hydrogenated rosinate and/or glyceryl hydrogenated rosinate). In some embodiments, the wax can include a non-rosinate base, such as modified colophonium and/or polycyclopentadiene. In some embodiments, the wax can include one or more sugars (e.g., dextrose, glucose, fructose, maltose, and combinations of these), such as honey wax or beeswax. The sugar wax can include additional components, e.g., glycerin, to modify the viscosity of the wax, citric acid as a formulation aid, fragrances, and/or colorants.

Generally, waxes are prepared in a heated reactor equipped with a motor and blades enabling good agitation and mixing. Resin-based hot waxes and wax components are typically formed by weighing the resin and other components into a reactor and heating the mixture, e.g., to about 120° C. with agitation. In some embodiments, paraffin wax is added to the mixture. Mixing is generally continued until a homogeneous solution is produced. The temperature is then increased (e.g., to a temperature of from about 135° C. to about 150° C., depending on the particular resin being used), and the temperature is maintained until the resin has dissolved. The mixture is cooled to 90° C., and any optional colorants and/or fragrances are added. At this point, the marine extracts are added. The mixture is then stirred to create a substantially homogeneous mixture, at which point the wax can be cooled to room temperature.

Warm sugar-based waxes are generally prepared by adding glycerin to a reactor at room temperature, and increasing the temperature to about 100° C. Citric acid, or other acids, e.g., ascorbic acid or dilute mineral acids such as dilute HCl or sulfuric acid, can be added to catalyze hydrolysis. The sugars are then added incrementally while the viscosity of the mixture is monitored. When addition of sugars is complete, heating is continued to bring the mixture to a boil (e.g., to a temperature of from about 130° C. to about 135° C.). Colorant can be added during this heating phase. When the mixture begins to boil, heating is stopped and the mixture is cooled to about 90° C. The marine extracts are then added, along with any other temperature-sensitive components (e.g., certain fragrances and/or colorants). Mixing continues until a substantially homogeneous mixture is obtained, at which point the wax can be cooled to room temperature.

Cold strip waxes are generally prepared by weighing the resin and wax components and liquid paraffin into a reactor and heating the mixture to about 135° C. with agitation. When a homogeneous mixture is achieved, the mixture is cooled to about 90° C., the marine extracts and any colorants and/or fragrances are added, and the wax is cooled to room temperature.

Waxes can irritate the skin by reason of the heat of the wax upon application (for hot waxes) and/or by virtue of the pain associated with the epilation process itself, namely, the pulling out of the hairs. Generally, however, waxes do not contain irritating chemicals, and do not require high pH's to be effective. As such, in certain embodiments, the wax can include a lower level of marine extracts while still providing beneficial effects to the skin being treated. For example, in certain embodiments, the wax can include a total level of marine extract (inclusive of all marine extracts in the skin care product) of at least about 1 ppm (e.g., at least about 5 ppm, 10 ppm, 50 ppm, 100 ppm, 500 ppm, 1000 ppm, 2000 ppm, 5000 ppm, 10,000 ppm, or 50,000 ppm) by weight and/or at most about 100,000 ppm (e.g., at most about 50,000 ppm, 10,000 ppm, 5000 ppm, 2000 ppm 1000 ppm, 500 ppm, 100 ppm, 50 ppm, 10 ppm, or 5 ppm) by weight.

Depilatory Creams and Gels

Depilatory creams and gels function to remove hair by chemical means, for example, by breaking down the hair to allow for its easy removal. Typically, one or more depilatory agents are included in a depilatory cream or gel, which is applied to the area of skin to be treated. A depilatory agent is an agent that can weaken the tensile strength of a hair shaft (e.g., that can penetrate the hair shaft and optionally break the hair shaft down, e.g., by breaking the disulfide bonds in the hair shaft). The hair thus loses its tensile strength, allowing for easy removal of the hair from at or below the skin surface.

Generally, the hair root is left undamaged. The cream and gel formulations are generally in the form of aqueous emulsions, optionally having an elevated pH to assist in the breakdown of hair. For example, in certain embodiments, the formulations can have a pH of no less than about 10.0 (e.g., no less than about 10.5, 11.0, 11.5, 12.0, 12.3, or 12.5) and/or no more than about 12.6 (e.g., no more than about 12.5, 12.3, 12.0, 11.5, 11.0, or 10.5). The emulsions can include one or more depilatory agents that function to break down hair. Exemplary depilatory agents include thioglycolic acid salts such as, for example, calcium thioglycolate, potassium thioglycolate, and lithium thioglycolate. Other exemplary depilatory agents include dithioerythritol, thioglycerol, thioglycol, thioxanthine, thiosalicyclic acid, N-acetyl-L-cysteine, lipoic acid, sulfides and polysulfides, NaHSO₃, Li₂S, Na₂S, K₂S, MgS, CaS, SrS, BaS, (NH₄)₂S, sodium dihydrolipoate, sodium 6,8-dithiooctanoate, NaSH, KSH, thioglycerol, 2-mercaptopropionic acid, 3-mercaptopropionic acid, thiomalic acid, glyceryl monothioglycolate, monoethanolamine thioglycolate, monoethanolamine thioglycolic acid, diammonium dithiodiglycolate, ammonium thiolactate, monoethanolamine thiolactate, thioglycolamide, homocysteine, cysteine, glutathione, dithiothreitol, dihydrolipoic acid, 1,3-dithiopropanol, thioglycolamide, glycerylmonothioglycolate, thioglycolhydrazide, keratinase, hydrazine sulphate, hydrazine disulphate, triisocyanate, guanidine thiglycolate, calcium thioglycolate, cysteamine, 2-mercaptoethanol, thiosalicylic acid, N-acetyl-L-cysteine, and cosmetically- and/or pharmaceutically-acceptable salts thereof. Alkali stannites (e.g., alkali stannites formed from stannous salts such as, e.g., SnCl2, SnSO4, SnO, SnBr2, and SnI2) can be employed, particularly when a stabilizer such as a polyhydroxycarboxylate anion is included as described in U.S. Pat. No. 4,111,653. Exemplary depilatory agents include those described in U.S. Pat. Nos. 6,306,380, 5,948,392, and 5,645,825.

The depilatory agents are typically incorporated into a cream base or a gel base. The cream or gel base is in certain embodiments composed primarily of water, and can include a thickening agent to provide sufficient thickness such that the cream or gel remains substantially in place upon application. Gels in particular generally include a thickening or gelling agent. The thickening agent can include, for example, a polymeric thickening agent. The polymeric thickening agent can include a cross-polymer (a polymer that is capable of cross-linking or that is cross-linked), for example, poly(methylvinylether/maleic anhydride) (PVM/MA) decadiene cross-polymer. Other thickening agents include Resassol VS, a blend of Deceth-7, PEG-40 hydrogenated castor oil and PPG-26-Buteth-26, and/or Biosaccharide Gum-1.

Cream bases are generally an emulsion, and can include cetearyl alcohol, 1PPG-15 stearyl ether, Ceteareth-20, and/or Garcinia Indica (kokum) seed butter. The creams are generally thickened by virtue of the energy (e.g., shear) imparted to the emulsion during processing.

The base can further include other components, e.g., colorants, fragrances, stabilizers, antioxidants, ingredients that aid in the depilatory process including, for example, bases (e.g., potassium hydroxide) and/or hair-swelling agents (e.g., urea), non-marine extract moisturizers (e.g., urea, silicates), antioxidants, preservatives, and/or processing aids (e.g., laureth-6 carboxylic acid, which can act as a solubilizer, e.g., to solubilize certain fragrances).

Use of the New Depilatory Compositions

In use, the depilatory compositions are generally applied as a relatively thick coating (e.g., up to about 2 mm thick) on the areas of skin where the user wishes to remove hair (e.g., the legs or axilla). The depilatory composition is left in situ for a sufficient length of time (e.g., 10 to 15 minutes) for the hair shaft to be chemically dissolved or damaged to the point at which it is easily removed. The composition is generally formulated to have a sufficiently high viscosity such that the composition does not flow away from the area to which it has been applied for at least the period of time in which it is left on the skin. After the depilatory composition has been left on the skin for a sufficient period of time, the composition and hair debris are wiped or washed off of the skin.

In some embodiments, the depilatory composition forms a solidified peelable coating, such that it can be removed by simply peeling the composition and hair debris from the skin. Such compositions can in certain embodiments include a polymer or polymer-forming compound and a compound (e.g., a cross-linking agent) that reacts with the polymer or polymer-forming compound to form the solidified peelable coating. Often, the polymer or polymer-forming compound and the reactive compound are included in separate parts of a two-part composition, which is mixed just prior to application to the skin or in successive applications on the skin itself. Exemplary depilatory compositions include those that are described in U.S. Pat. No. 5,026,542.

Depilatory products generally contain chemicals that can, in some circumstances, irritate and/or damage skin, e.g., under conditions of normal use. Additionally, many depilatory compositions have a pH high enough to irritate the skin. Thus, the depilatory products can in certain embodiments include a relatively higher level of marine extracts than the waxes described above to prevent, reduce and/or alleviate skin irritation and/or damage resulting from the depilatory product. For example, in certain embodiments, the depilatory product can have a total level of marine extract (inclusive of all marine extracts in the skin care product) of at least about 50 ppm (e.g., at least about 100 ppm, 500 ppm, 1000 ppm, 5000 ppm, 10,000 ppm, 50,000 ppm, or 100,000 ppm) by weight and/or at most about 200,000 ppm (e.g., at most about 100,000 ppm, 50,000 ppm, 10,000 ppm, 5000 ppm, 1000 ppm, 500 ppm, or 100 ppm) by weight.

In still other embodiments, the depilatory product can be in the form of an aerosol (e.g., a bag-on-valve can). The depilatory product is sprayed onto an area of skin to be treated, where it remains for a period of time sufficient for the depilatory action to occur.

Formulations delivered from aerosol cans (e.g., bag-on-valve type aerosol cans) generally do not significantly change in viscosity when they are dispensed. The formulations are designed to have a sufficiently high viscosity so as to adhere to the skin and hair. In certain other embodiments, which are the subject of a separate patent, the formulation may be dispensed from a bag-on-valve can type of aerosol to produce a gel, which transition to a foam as dispersed propellant volatilizes on exposure to body temperature. The viscosity and wetting characteristics of the resulting foam allow the depilatory product to remain on the skin.

Pre- and Post-Depilatory Applications

In some embodiments, marine extracts are included in a composition that is applied just prior to, or just after, the use of a depilatory and/or epilatory product, for example, to reduce, prevent, or treat irritated and/or damaged skin. The compositions can be in the form of a gel, an oil, or a lotion, or can be included in a solution contained in a wipe (e.g., a wipe used to clean the skin prior to and/or following the removal of hair).

In certain embodiments, a pre-depilatory composition is applied, e.g., prior to applying a wax or a depilatory product. The pre-depilatory composition can in certain embodiments be in the form of a cream, a gel, a lotion, or a cleansing wipe. The composition includes marine extracts in any of the formats disclosed herein. The pre-depilatory compositions can include a cleaning agent, such as alcohol, e.g., a denatured alcohol, such that they rapidly evaporate from the skin surface, leaving the skin ready to accept a depilatory composition. They can also include a numbing agent, such as an aromatic anesthetic, e.g., menthol, eucalyptol, menthyl lactate, menthone glycerol ketal, WS-23 (2-Isopropyl-N,2,3-trimethylbutyramide), WS-3 (N-Ethyl-p-menthane-3-carboxamide), or WS-5 [Ethyl 3-(p-menthane-3-carboxamido)acetate]; or a salicylate, such as methyl or sodium salicylate. The compositions can further include other optional components having skin-enhancing effects.

The pre-depilatory compositions include one or more (e.g., two or more, three or more, four or more, or five) marine extracts selected from those described herein, to provide skin-enhancing benefits. For example, a marine extract blend can be included at levels of at least about 0.02% (e.g., at least about 0.04%, 0.06%, 0.08%, 0.10%, 0.12%, 0.14%, 0.16%, 0.18%, 0.20%, 0.25%, 0.3%, 0.4%, 0.5%, 0.6%, 0.7%, 0.8%, 0.9%, or 1.0%) by weight of the final product and/or at most about 2.0% (e.g., at most about 1.0%, 0.9%, 0.8%, 0.7%, 0.6%, 0.5%, 0.4%, 0.3%, 0.25%, 0.2%, 0.18%, 0.16%, 0.14%, 0.12%, 0.10%, 0.08%, 0.06%, or 0.04%) by weight of the composition.

In certain embodiments, a post-depilatory composition is applied, e.g., after applying a wax or a depilatory product. The post-depilatory composition can also be in the form of a cream, a gel, a lotion, or a cleansing wipe, and also includes marine extracts in any of the formats disclosed herein, which can be at the same weight percentages as described for the pre-depilatory compositions. Typically, a post-depilatory composition will have a non-alcohol base (e.g., will have a water or oil base, e.g., mineral oil) to avoid drying and/or burning the freshly-depilated skin.

Non-Epilatory and/or Depilatory Skin Care Products

In certain embodiments, marine extracts (e.g., a marine extract blend comprising one or more, two or more, three or more, or four or more of Entermorpha Compressa extract, Fucus Vesiculosus extract, Porphyra Umbilicalis extract, Undaria Pinnatifida extract, and Lithothamnium Calcareum extract) can be incorporated into non-epilatory and/or depilatory skin care products associated with shaving (e.g., shaving creams, gels, soaps, shaving aid strips such as are often included on razor heads, after-shave gels, balms, lotions, and/or splashes).

In other embodiments, marine extracts (e.g., a marine extract blend comprising one or more, two or more, three or more, or four or more of Enteromorpha Compressa extract, Fucus Vesiculosus extract, Porphyra Umbilicalis extract, Undaria Pinnatifida extract, and Lithothamnium Calcareum extract) can be incorporated into skin care products not necessarily associated with hair removal (e.g., skin creams, moisturizers, sunscreens, creams and gels for use in relief of sunburns, and/or lip balms).

### EXAMPLES

The invention is further described in the following examples, which do not limit the scope of the invention described in the claims.

### Example 1

Two embodiments of a marine extract blend are provided in Table 1 below, with all percents being weight percents. Marine Extract Blend A would typically be used in sugar-based waxes, pre-depilatory wipes, and/or in post-depilatory lotions or oils, while Marine Extract Blend B would typically be used in wax strips, hot waxes, and post-depilatory lotions or oils.

TABLE 1

| | Marine | Marine | |
|---|---|---|---|
| | Extract | Extract | |
| Ingredient | Blend A | Blend B | Function |
| Glycerin | 40-60% | — | Diluent |
| Water | 40-60% | — | Diluent |
| Mineral Oil | — | 94.8-99.7% | Diluent |
| Fucus Vesiculosus Extract | 0.5-1.0% | 0.5-1.0% | |
| Enteromorpha Compressa | 0.5-1.0% | 0.5-1.0% | |
| Extract | | | |
| Porphyra Umbilicalis | 0.5-1.0% | 0.5-1.0% | |
| Extract | | | |
| Undaria Pinnatifida | 0.5-1.0% | 0.5-1.0% | |
| Extract | | | |
| Lithothamnium Calcarum | 0.5-1.0% | 0.5-1.0% | |
| Extract | | | |
| Potassium Sorbate | 0.2% | — | Preservative |
| Sodium Benzoate | 0.2% | — | Preservative |
| Antioxidants (soybean oil | — | 0.2% | Antioxidant |
| and rosemary leaf extract) | | | |

### Example 2

An embodiment of a depilatory sugar wax formulation is provided in Table 2 below. This embodiment includes Marine Extract Blend A from Example 1, above.

TABLE 2

| Ingredient | Weight % | Function |
|---|---|---|
| Dextrose | 86.4 | Wax base |
| Glycerin | 11.6 | Viscosity modifier |
| Citric acid | 1.5 | Formulation aid |
| Mica and titanium dioxide | 0.4 | Mica: sparkles; TiO₂ allows for |
| | | color |
| Parfum | 0.1 | Fragrance |
| Marine Extract Blend A | 0.01 | Marine extract blend |
| CL 42090 | 0.001 | Pigment |

### Example 3

An embodiment of a depilatory gel formulation including marine extracts is provided in Table 3 below. This embodiment includes Marine Extract Blend B from Example 1, above. This formulation can function as an aerosol.

TABLE 3

| Ingredient | Weight % | Function |
|---|---|---|
| Water | 62.9 | Diluent |
| Potassium Thioglycolate, | 15 | Active depilatory ingredient |
| water | | |
| Urea | 5 | Swells hair, moisturizes skin |
| Sodium Silicate | 5 | pH adjuster, skin moisturizer |
| Potassium Hydroxide | 5.4 | pH adjuster, aids in breaking |
| | | down chemical bonds in hair |
| PVM/MA Decadiene | 3.2 | Polymer thickening agent |
| Crosspolymer | | |
| Glycerin | 2 | Moisturizer |
| Parfum | 0.75 | Fragrance |
| Laureth-6 Carboxylic Acid | 0.5 | Fragrance solubilizer |
| Sodium Gluconate | 0.2 | Prevents color change in |
| | | thioglycolate formulations |
| Marine Extract Blend B | 0.1 | Marine extract blend |
| C.I. 474620 (water, glycerin, | 0.001 | Green pigment |
| sodium lauryl sulfate, | | |
| o-phenylphenol) | | |
| C.I. 74160 (water, glycerin, | 0.001 | Blue pigment |
| sodium lauryl sulfate, | | |
| o-phenylphenol) | | |

### Example 4

An embodiment of a pre-depilatory solution including marine extracts is provided in Table 4 below. This embodiment includes Marine Extract Blend B from Example 1, above.

TABLE 4

| Ingredient | Weight % |
|---|---|
| Denatured alcohol | 99.3 |
| Eucalyptol | 0.4 |
| Menthol | 0.1 |
| Marine Extract Blend B | 0.1 |
| Methyl Salicylate | 0.05 |
| CI 42090 | 0.05 |

### Example 5

An embodiment of a post-depilatory oil formulation including marine extracts is provided in Table 5 below. This embodiment includes Marine Extract Blend B from Example 1, above.

TABLE 5

| Ingredient | Weight % |
|---|---|
| Paraffinum liquidum | 89.4 |
| Isopropyl myristate | 10.0 |
| Perfume | 0.3 |
| Marine Extract Blend B | 0.1 |
| Phenoxyethanol, methylparaben, propylparaben, | 0.1 |
| butylparaben, and isobutylparaben mixture | |
| Tetradibutyl pentaerithrityl hydroxyhydrocinnamate | 0.1 |

### Example 6

An embodiment of a post-depilatory cream formulation including marine extracts is provided in Table 6 below. This embodiment includes Marine Extract Blend B from Example 1, above.

TABLE 6

| Ingredient | Weight % |
|---|---|
| Water (deionized) | 81.2 |
| C₁₂-C₁₅ Alkyl Benzoate | 2.0 |
| Cetyl Alcohol | 2.0 |
| Glycerin | 2.0 |
| Steareth-21 | 2.0 |
| Propylene Glycol Dicaprylate/Dicaprate | 2.0 |
| Paraffinum Liquidum | 2.0 |
| Steareth-2 | 2.0 |
| Propylene Glycol | 0.77 |
| Phenoxyethanol, Methylparaben, Propylparaben, | 0.7 |
| Butylparaben, Isobutylparaben mixture | |
| Dimethicone | 0.5 |
| Perfume | 0.3 |
| Imidazolidinyl Urea | 0.1 |
| Carbomer | 0.1 |
| Bisabolol | 0.1 |
| Marine Extract Blend B | 0.1 |
| Lactic Acid | 0.09 |
| Triethanolamine | 0.074 |
| Lemon Extract | 0.03 |
| CI 42090 | 0.001 |

### OTHER EMBODIMENTS

It is to be understood that while the invention has been described in conjunction with the detailed description thereof, the foregoing description is intended to illustrate and not limit the scope of the invention, which is defined by the scope of the appended claims, and that various modifications may be made without departing from the spirit and scope of the invention. Other aspects, advantages, and modifications are within the scope of the following claims.

What is claimed is:

1. A composition comprising a depilatory or epilatory agent and at least two marine extracts selected from the group consisting of an Enteromorpha Compressa extract, a Fucus Vesiculosus extract, a Porphyra Umbilicalis extract, an Undaria Pinnatifida extract, and a Lithothamnium Calcareum extract.

2. The composition of claim 1, comprising a Fucus Vesiculosus extract and a Porphyra Umbilicalis extract.

3. The composition of claim 2, further comprising an Enteromorpha Compressa extract.

4. The composition of claim 3, further comprising an Undaria Pinnatifida extract.

5. The composition of claim 1, comprising each of an Enteromorpha Compressa extract, a Fucus Vesiculosus extract, a Porphyra Umbilicalis extract, a Undaria Pinnatifida extract, and a Lithothamnium Calcareum extract.

6. The composition of claim 1, wherein each marine extract is present at a level that is no less than about 50% of any other marine extract.

7. The composition of claim 1, further comprising a diluent selected from the group consisting of water, glycerin, mineral oil, and mixtures thereof.

8. The composition of claim 1, wherein each marine extract is present at a level of at least about 5 ppm.

9. The composition of claim 1, wherein the marine extracts, in combination, are present at a level of at least about 10 ppm.

10. The composition of claim 1, wherein the marine extracts, in combination, are present at a level of at least about 25 ppm.

11. The composition of claim 1, wherein the depilatory agent comprises a thioglycolic acid salt.

12. The composition of claim 1, wherein the depilatory agent comprises potassium thioglycolate.

13. The composition of claim 1, wherein the pH of the composition is from about 10.0 to about 12.6.

14. The composition of claim 1, wherein the depilatory agent comprises an alkali stannite.

15. The composition of claim 1, further comprising urea.

16. The composition of claim 1, further comprising at least one of a pigment, a fragrance, and a moisturizer that is not a marine extract.

17. The composition of claim 1, further comprising a cross-polymer.

18. The composition of claim 17, wherein the cross-polymer is poly(methylvinylether/maleic anhydride) decadiene.

19. The composition of claim 1, wherein the epilatory agent is a wax.

20. The composition of claim 19, wherein the wax comprises a sugar-based wax.

21. The composition of claim 20, wherein the sugar wax comprises dextrose.

22. The composition of claim 19, wherein the wax comprises a hydrogenated rosinate.

23. The composition of claim 19, wherein each marine extract is present at a level of at least about 0.5 ppm.

24. The composition of claim 19, wherein the marine extracts, in combination, are present at a level of at least about 1 ppm.

25. The composition of claim 19, wherein the marine extracts, in combination, are present at a level of at least about 2.5 ppm.

26. The composition of claim 19, further comprising at least one of a pigment, a fragrance, a viscosity modifier, and a moisturizer that is not a marine extract.

27. An epilatory composition comprising:
a marine extract blend comprising at least two of an Enteromorpha Compressa extract, a Fucus Vesiculosus extract, a Porphyra Umbilicalis extract, a Undaria Pinnatifida extract, and a Lithothamnium Calcareum extract;
a sugar wax; and
glycerin.

28. The composition of claim 27, comprising from about 0.005% to about 0.02% by weight marine extract blend, from about 75% to about 90% by weight dextrose, and from about 5% to about 15% by weight glycerin.

29. The composition of claim 27, wherein the marine extract blend comprises between about 0.5% and about 1% by weight of each of an Enteromorpha Compressa extract, a Fucus Vesiculosus extract, a Porphyra Umbilicalis extract, an Undaria Pinnatifida extract, and a Lithothamnium Calcareum extract.

30. The composition of claim 27, wherein the marine extract blend further comprises mineral oil.

31. The composition of claim 27, wherein the marine extract blend further comprises an antioxidant.

32. A depilatory composition comprising:
a marine extract blend comprising at least two of an Enteromorpha Compressa extract, a Fucus Vesiculosus extract, a Porphyra Umbilicalis extract, a Undaria Pinnatifida extract, and a Lithothamnium Calcareum extract;
a thioglycolate salt;
urea;
sodium silicate; and
water.

33. The composition of claim 32, comprising from about 0.05% to about 0.2% by weight marine extract blend, from about 10% to about 20% by weight thioglycolate salt, from about 2% to about 8% urea, and from about 2% to about 8% sodium silicate.

34. The composition of claim 32, further comprising from about 1% to about 5% of a cross-polymer.

35. The composition of claim 32, wherein the marine extract blend comprises between about 0.5% and about 1% by weight of each of an Enteromorpha Compressa extract, a Fucus Vesiculosus extract, a Porphyra Umbilicalis extract, an Undaria Pinnatifida extract, and a Lithothamnium Calcareum extract.

36. The composition of claim 32, wherein the marine extract blend further comprises glycerin.

37. A composition comprising a diluent and a marine extract blend comprising at least two of an Enteromorpha Compressa extract, a Fucus Vesiculosus extract, a Porphyra Umbilicalis extract, a Undaria Pinnatifida extract, and a Lithothamnium Calcareum extract.

38. The composition of claim 37, wherein the marine extract blend comprises between about 0.5% and about 1% by weight of each of the Enteromorpha Compressa extract, the Fucus Vesiculosus extract, the Porphyra Umbilicalis extract, the Undaria Pinnatifida extract, and the Lithothamnium Calcareum extract.

39. The composition of claim 37, wherein the diluent comprises mineral oil.

40. The composition of claim 37, further comprising an antioxidant.

41. The composition of claim 37, wherein the diluent comprises water, glycerin, or both water and glycerin.

42. The composition of claim 37, further comprising a preservative.

43. The composition of claim 37, wherein the diluent comprises a cleaning agent and further comprising a numbing agent.

44. The composition of claim 43, wherein the cleaning agent comprises alcohol and the numbing agent comprises an aromatic anesthetic or a salicylate.

45. The composition of claim 44, wherein the aromatic anesthetic is menthol, eucalyptol, menthyl lactate, menthone glycerol ketal, WS-23 (2-Isopropyl-N,2,3-trimethylbutyramide), WS-3 (N-Ethyl-p-menthane-3-carboxamide), or WS-5 [Ethyl 3-(p-menthane-3-carboxamido)acetate].

46. The composition of claim 44, wherein the salicylate is methyl salicylate or sodium salicylate.

47. A composition of claim 37, wherein the Enteromorpha Compressa extract comprises an oligosaccharide, the Fucus Vesiculosus extract comprises an alginate and organic iodine, the Porphyra Umbilicalis extract comprises an amino acid, the Undaria Pinnatifida extract comprises a peptide and a polysaccharide, and the Lithothamnium Calcareum extract comprises a mineral salt.

48. The composition of claim 37, wherein the composition comprises at least three marine extracts.

49. The composition of claim 37, wherein the composition comprises at least four marine extracts.

50. The composition of claim 37, comprising each of an Enteromorpha Compressa extract, a Fucus Vesiculosus extract, a Porphyra Umbilicalis extract, a Undaria Pinnatifida extract, and a Lithothamnium Calcareum extract.

51. A method of making the composition of claim 37, the method comprising
obtaining the two or more marine extracts;
obtaining the diluent; and
mixing the marine extracts into the diluent at a temperature below 90° C.

52. A method of soothing the skin, the method comprising applying the composition of claim 37 to the skin.
